(19) United States
(12) Patent Application Publication
(10) Pub. No.: US 2005/0149378 A1
(43) Pub. Date: Jul. 7, 2005

(54) System and method for management of clinical supply operations

(76) Inventors: Keneth K. Cyr, Liberty, MO (US); Barry C. Dyer, Overland Park, KS (US)

Correspondence Address: SHOOK, HARDY & BACON L.L.P., 2555 GRAND BOULEVARD, KANSAS CITY, MO, 64108-2613, US

(21) Appl. No.: 10/750,209

(22) Filed: Jan. 2, 2004

(51) Int. Cl. G06F 17/60

(52) U.S. Cl. 705/10

(57) ABSTRACT

A system and related techniques generate and track clinical supply orders, policies, individual or aggregate consumption and automated billing, ordering and fulfillment functions via an integrated clinical platform. A patient supply record may be generated for individual patients which traces the consumption of surgical, disposable, pharmaceutical and other supplies throughout a medical encounter or series of encounters or treatment. Physician supply preferences may be aggregated and analyzed to derive value leverage, for instance through volume pricing or conforming department supplies to standard practices. In implementations the supply selections of a given doctor or department or for specified categories of procedure may be analyzed in terms of ultimate patient outcomes resulting from use of those clinical supplies to identify best results and optimize other aspects of clinical operation. A supply chain engine may communicate with supply tracking resources to automatically trigger orders for different supplies, for instance through enterprise resource planning (ERP) or other channels. In embodiments the supplies and materials so ordered may be automatically fulfilled or delivered from the vendor side, for instance based on quantity threshold or closest or most efficient delivery points.

### CROSS-REFERENCE TO RELATED APPLICATION

Not applicable.

### STATEMENT REGARDING FEDERALLY SPONSORED RESEARCH OR DEVELOPMENT

Not applicable.

### FIELD OF THE INVENTION

The invention relates to the field of management information systems in the medical industry, and more particularly to an integrated platform to capture clinical supply consumption at the individual patient and procedure level, and analyze overall clinical consumption patterns in a hospital or other facility to optimize supply selections, patient outcomes, costs, delivery schedules or other operational criteria.

### BACKGROUND OF THE INVENTION

Hospitals and other clinical facilities face the management task of managing the effective delivery of health services while containing the overall costs of their clinical operations. Administrators at a large hospital may have to track inventory, manage ordering and coordinate billing for a vast array of medical supplies in the clinical environment. Supplies and material from surgical tools, implants, electronic monitoring or diagnostic equipment, gowns, gloves, pharmaceuticals, disposable material such as tissues, bandages and a host of other supplies must be monitored, stored and requisitioned in a timely manner to ensure the smooth operation of surgical, radiological, emergency and other departments and facilities.

Certain commercially available management information systems (MISs) exist which may permit hospital administrators to select, purchase and monitor supplies and material for their various operations. However, those platforms suffer from disadvantages of various types. For one, in commercial systems clinical supply consumption may be tracked or monitored, but only at a department or facility-wide level. For instance the director of a surgical unit may be able to review how many scalpels or stents his or her unit consumed last month, or whether a new order of tracheal tubing needs to be ordered for emergency room staff. However, those MIS platforms permit the administrator to view and manage clinical supplies only at that aggregate level, without associating supply consumption to specific patients, doctors, supply vendors, procedures or other types or details of individual encounters or care. Cost recovery on a patient basis, for instance to attempt to assign costs of supplies consumed during a specific cardiac surgery or radiology scan, can therefore be difficult. Billing departments may be left with no alternative other than simply averaging costs of those supplies over all patients, or assigning that clinical consumption to other cost centers within the organization. This may lead to cost distortions for insurance payment and other purposes.

The lack of ability to accurately track medical supply consumption to the lowest level of clinical detail also has an impact on other aspects of hospital or other clinical operations. For one, many clinical units solicit the supply preferences of doctors and other care providers through a record of stored supply selections, sometimes called a preference card. So for example a given orthopedic surgeon may be known to prefer scalpels, laparoscopes or other supplies or instruments of certain types, sizes or make, which may permit administrators to order and stock those types of supplies for that provider and his or her operating room. However, in general those same administrators may have no view on the collective supply preferences of their medical staff. Facilities may therefore have to assign managers to manually track consumption patterns to reach even a rudimentary analysis of supply selections and costs, a task which may require several person-weeks for even just one item.

Lacking that type of insight, administrators may not be able to determine whether the medical staff of a unit or department shows a trend toward certain types of supplies, vendors or technologies. Persons responsible for supply procurement may as one consequence not be able to negotiate the most favorable purchase arrangements with vendors or others without knowledge of that type of data. Uniform supply policies may likewise be more difficult to formulate, revise or enforce when the clinical preferences of a clinical unit can not be accurately known or leveraged.

Moreover, clinical managers may certainly have no ability to associate given supply selections with actual clinical outcomes which result from using those supplies. So if the widespread use of a given type of surgical stent or hip replacements has been yielding improved patient results within the organization, managers may not be able to discern that positive trend. They similarly may not be able to see that certain surgical or other supplies work best in combination, or other higher level patterns or trends. Personnel performing those types of reviews may conversely not be able to drill down to individual patients for whom a supply item was used, or isolate which type of procedure the item was used in. Clinical administrators may therefore may not be able to leverage or standardize the use of advantageous products in clinical supply, billing or other policies.

Collective supply activities can not be effectively or comprehensively managed on today's information platforms, on the procurement side as well. While many hospitals and other facilities keep computerized records of clinical supplies present and available in given departments, no effective or integrated mechanism exists to order and replenish those supplies on demand. In fact, some emergency room and other operations simply rely upon manual reordering by staff when an inventory cart, supply cabinet or other dispensing locations are low on supplies. Even when database tools permit managers a quantitative view on remaining inventory or available supplies, requisitioning those supplies is still often left to a manual ordering and fulfillment process. This does not permit the organized or rationalized management of supply purchases, for instance to batch purchases between departments, seek volume discounts from vendors, time purchases for known peak periods, order from closest or most efficient suppliers or take other active steps to manage the purchase and delivery of necessary material. Certainly existing platforms do not leverage the possibility of establishing a supply chain network in which supply orders may be automatically generated based on actual clinical events and the effect of these events on inventory states, or automatically fulfilled via a vendor communications channel and electronic billing arrangement. Other problems in current clinical supply platforms and practices exist.

### SUMMARY OF THE INVENTION

The invention overcoming these and other problems in the art relates in one regard to a system and method for management of clinical supply operations, in which a management platform establishes clinical preferences, tracks all phases of clinical consumption, associates that consumption to individual patients and permits the generation of reports at all available levels of detail for clinical administrators and others. In embodiments, a patient supply record may be instantiated for individual patients which cumulatively tracks the supplies, pharmaceuticals and other physical material consumed or used in that patient's care, permitting cost association to specific encounters as well as higher-level reports. According to the invention in another regard, the selection of given classes of clinical supplies by physicians and other care providers may be aggregated and evaluated for clinically driven value analytics such as volume pricing, vendor support and other operational criteria. In embodiments, the use and consumption of surgical, therapeutic and other supplies may be analyzed in terms of ultimate patient outcomes, to thereby inform clinical administrators about desirable material and supplies, and practices used with those supplies. In further embodiments, the stock of clinically available supplies may be monitored based on consumption during documented clinical events and tracked in a realtime or near realtime basis to automatically generate supply orders, down to the level of individual clinical events or encounters if necessary. In other embodiments, the procurement orders so generated may be communicated to a supply vendor for automatic or semiautomatic fulfillment and delivery via a supply chain engine. According to the invention in one regard, all aspects of clinical supply specifications and preferences, purchasing, delivery and therapeutic use may be tracked via the inventive platform, permitting optimized supply selection, patient outcomes and more accurate cost accounting for that materiel.

### BRIEF DESCRIPTION OF THE DRAWINGS

FIG. 1 illustrates an architecture for managing supply selection policy, according to an embodiment of the invention.

FIG. 2 illustrates an architecture for the tracking of patient supply records, according to an embodiment of the invention.

FIG. 3 illustrates the generation of a patient supply record, according to embodiments of the invention.

FIG. 4 illustrates an architecture for generating outcomes-based analytics based upon clinical supply usage, according to an embodiment of the invention.

FIG. 5 illustrates analytic reports at different levels of supply-based outcomes detail, according to an embodiment of the invention.

FIG. 6 illustrates an architecture in which a supply chain engine and related tracking resources may operate, according to an embodiment of the invention.

FIG. 7 illustrates an architecture in which a supply fulfillment engine may operate, according to embodiments of the invention.

FIG. 8 illustrates a flowchart of overall supply preference management, according to embodiments of the invention.

FIG. 9 illustrates a flowchart of the analysis of patient supply selections in terms of patient outcomes, according to embodiments of the invention.

FIG. 10 illustrates a flowchart of overall supply ordering and delivery management, according to embodiments of the invention.

### DETAILED DESCRIPTION OF EMBODIMENTS

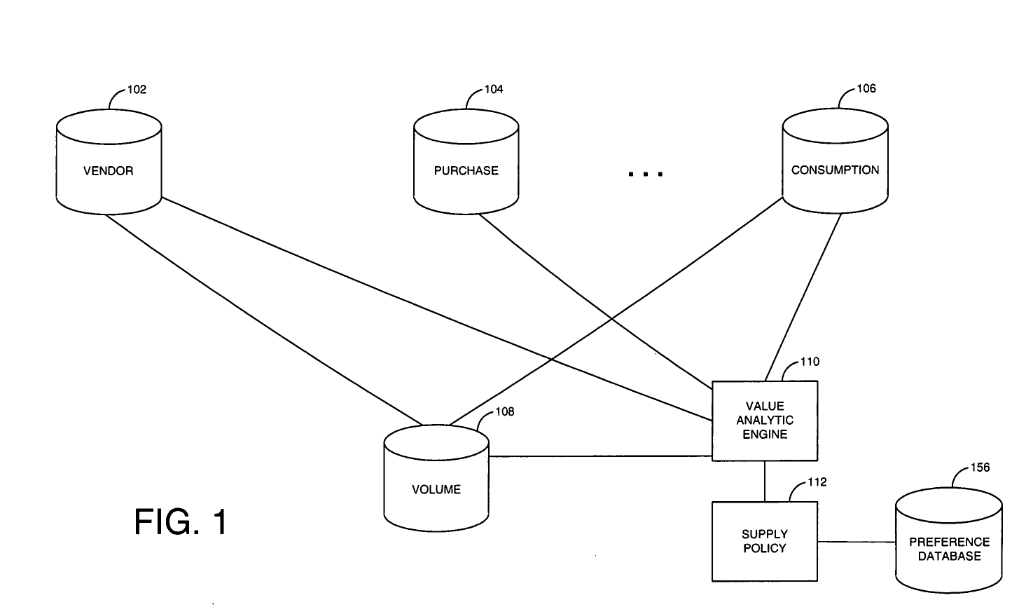

FIG. 1 illustrates an architecture in which a system and method for management of clinical supply operations may operate, according to an embodiment of the invention. As shown in that figure, different metrics which track different aspects of a hospital or other clinical operation may be collected and stored to various data stores. That captured data may include, for example, vendor or manufacturer data stored to a vendor database 102, purchase or transaction data for supplies and materials stored to a purchase database 104, and data regarding supplies which are picked or used from available supply which is stored to consumption database 106. Other data types and data stores are possible.

According to embodiments of the invention, each of vendor database 102, purchase database 104 and consumption database 106 may be linked to a volume database 108 which associates clinical supply purchases with vendor identifiers, clinical use and other variables to capture cost data including cumulative purchase, consumption and other rates for various clinical supplies. The clinical supplies tracked and managed by embodiments of the invention may be or include any surgical, medical, diagnostic or other instruments, equipment, pharmaceutical or other clinically related disposable or non-disposable items, such as, for example, surgical instruments such as scalpels, forceps, catheters, laparoscopes, joint, bone, dental or other implants or others, intravenous lines, saline solution, blood serum, syringes, laboratory supplies such as fluid sample cartridges, assay solution or other material, diagnostic material such as X-ray film, pharmaceuticals such as antibiotics or analgesics or other prescription or non-prescription medications or treatments, protective clothing such as gowns, masks or others or any other clinically related material. A hospital or other administrator may therefore be able to view the “run” or consumption rate for supplies of surgical instruments or blood serum orders, or calculate the total cost of splints, bandages or other disposable or other material at that clinical site for the year.

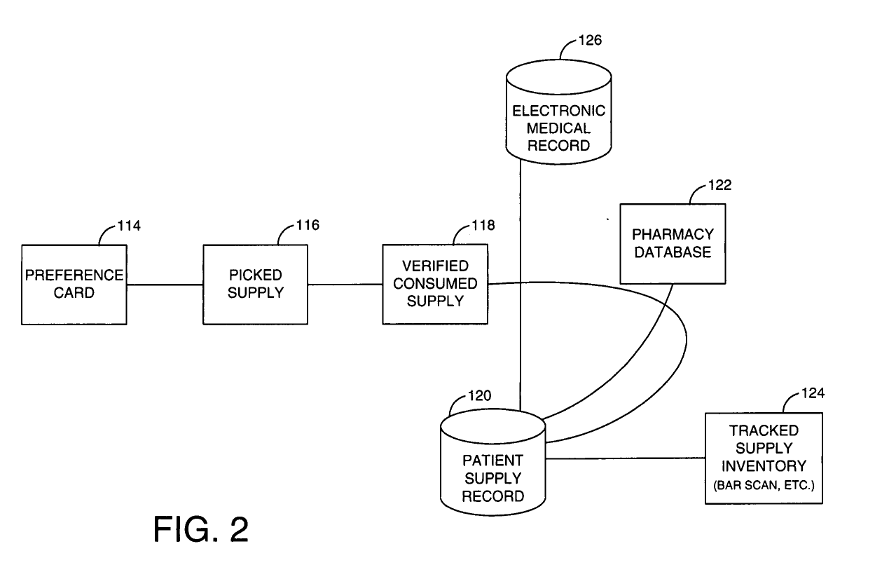

As likewise illustrated in FIG. 1, the selection and consumption of clinical supplies at a hospital or other clinically related site may be guided by the supply selections of physicians and other care providers. More specifically as shown each care provider may select preferred surgical instruments, anesthesiology drugs or equipment, implants, pharmaceuticals, stethoscope, thermometer or other diagnostic instruments or other supplies, material, pharmaceuticals or other hardware, disposables or other material related to clinical care. In embodiments each physician or other care provider may select preferred supply choices on a physical or electronic preference card (114 in FIG. 2) whose selections are recorded in a preference database 156.

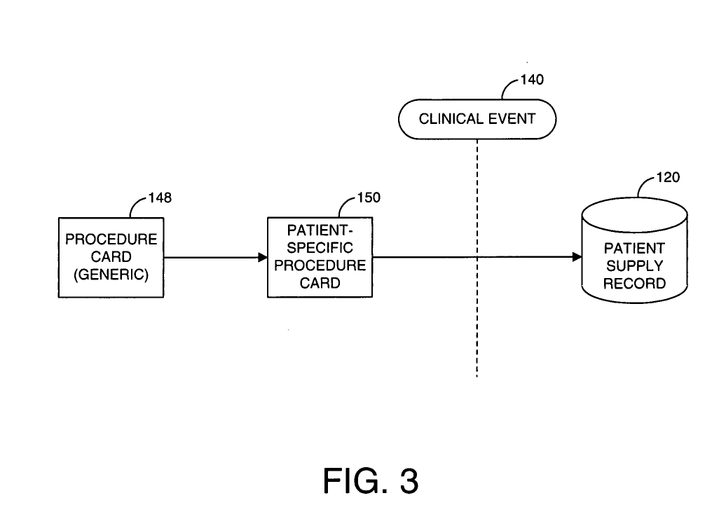

When a clinical event (140 in FIG. 3) such as a consultation, evaluation, surgery, X-ray imaging or other patient encounter or instance of treatment takes place, the preference database 156 may be accessed to determine the preferred clinical material for the given type of procedure for the one or more care providers attending the patient. The clinical documentation for the clinical event records the clinical supplies which are actually used or consumed during the clinical event may be recorded to consumption database 106, to indicate or update resulting used and unused, substituted or other supply inventory.

According to the invention in one regard, the volume database 108, consumption database 106 and other data sources may be likewise linked to a value analytic engine 110, which for instance may be or include a structured query language (SQL) engine to query the supply-related and other data populating those data stores. In embodiments the value analytic engine 110 may analyze the overall preference selections for a surgical, cardiac or other unit and determine for example which percentage of surgeons are choosing a particular stent model manufactured by a certain vendor versus another vendor. Aggregate consumption patterns may be broken down along other clinical lines as well, for instance according to practice area, physician seniority or other groupings.

A medical director, chief surgeon or other administrator or manager may therefore discern the trends and patterns of supply consumption of their facility at any available level of clinical detail. Viewing that data may aid the administrator or other user to make more effective supply choices in value and other terms. For example the user may see that the hospital or other clinically related site currently uses a certain number of laser cauterizing units per year, whereas the manufacturer may give a discount on pricing or service or maintenance benefits if the facility purchases 10% more.

This may lead the administrator or other to adopt a standardized selection for that clinical supply for one or more units, or all relevant clinical units in the facility to gain that global benefit for the organization. In embodiments this or other types of supply standardization may be expressed in a supply policy 112, which may be communicated to physicians and others in the facility. In embodiments the supply policy may be communicated to the preference database 156, for instance to automatically update the preference card 114 of one or more care provider so that supply selections or supply defaults may be uniformly deployed. In this manner the clinically driven value analysis guided by reports generated via value analytic engine 110 may permit a more rationalized or cost effective set of supply selections for the hospital or other clinically related site.

FIG. 2 illustrates a platform for management of clinical supply operations, according to embodiments of the invention in another regard. It may be noted that while available medical management information systems permit hospital administrators to track various aspects of supply consumption, dollar volumes of purchasing and other aspects of supply management, those tools lack the ability to track supply usage beyond the unit-wide or other macro level.

According to embodiments of the invention illustrated in FIG. 2, analytic views on supply selection, consumption and cost may be extended to the individual patient level, or lower to single encounters or other micro clinical details. According to embodiments of the invention as shown, supply details of clinical treatment and encounters at the level of an individual patient may be recorded and tracked in a patient supply record 120, which comprehensively traces consumed supplies and materiel to individual patients during the course of their entire medical care.

More specifically and as illustratively shown, when an individual patient experiences a clinical event 140, the preference card 114 for attending physicians or other care providers may be consulted to make appropriate surgical, pharmaceutical, diagnostic or other supplies available for the given procedure or treatment. During the occurrence of the clinical event 140, the physicians or other care providers may make on-the-spot selections from the clinically available supplies and use that picked supply 116 to deliver care. During or after the clinical event 140, the picked supply 116 and other data may be stored in a verified consumed supply record 118 which records items and quantities of supplies consumed during the clinical event 140. In embodiments the verified consumed supply record 118 may likewise link to a pharmacy database 122 to record pharmaceuticals prescribed or administered, if appropriate.

According to the invention in one regard, the verified consumed supply record 118 and other data may be communicated to patient supply record 120 to be recorded and associated with the given patient and their individual supply profile. The patient supply record 120 may in embodiments likewise be linked to a tracked supply inventory 124 which may, for example, record or encode supplies ordered or used via a bar code scanner, radio frequency identification (RFID) tag, manual entry or other coding. The data entered into patient supply record 120 may thereby be reconciled or updated with physical supply codes captured on the clinical floor, and in embodiments the tracked supply inventory 124 may also be used to trigger automated supply ordering or other processing, as for example described otherwise herein. Patient supply record 120 may also link to an electronic medical record (EMR) database 126, to access and store clinical information related to the patient's medical condition and care.

Because the patient supply record 120 amalgamates links and content from a variety of clinical databases which capture various aspects of supply identification and use, that record may reflect a complete record of a patient's supply consumption during the entire course of clinical treatment or care. Because that repository is comprehensive, administrators and others may generate reports off of patient supply record 120 to determine exactly how much of a given antibiotic, stent, intravenous supply or line or other supply, pharmaceutical or material has been used or consumed in that patient's overall treatment.

Associated supply costs may therefore be accurately attributed to a given patient and their exact clinical encounters and treatments, leading to more accurate source data for billing, insurance, supply reordering, clinical evaluation and other administrative or clinical functions. While the invention permits views into individual patients in patient supply record 120, it may be noted that administrators and others may still generate aggregate reports on costs or consumption patterns for groups of patients from one or more patient supply record 120, when desired.

FIG. 3 schematically illustrates the generation of a patient supply record 120, which may for instance be generated following or in conjunction with one or more patient encounters or at other times, according to which a physician or other care provider may consult or create a procedure card 148, for instance detailing necessary supplies for a standard carpal tunnel procedure. The physician or other care provider or other may then derive a patient-specific procedure card 150 to encapsulate necessary clinical supplies for that particular patient and their scheduled procedure, for instance taking into account the patient's age, physical condition, allergies or other factors. After the clinical event 140 is carried out, for instance at a hospital or other clinically related site, the patient supply record 120 may be initiated or updated with data from the patient-specific procedure card 150, or from other sources. The patient supply record 120 may thereby trace the supplies and material actually consumed by an individual patient for clinical, billing, insurance and other purposes.

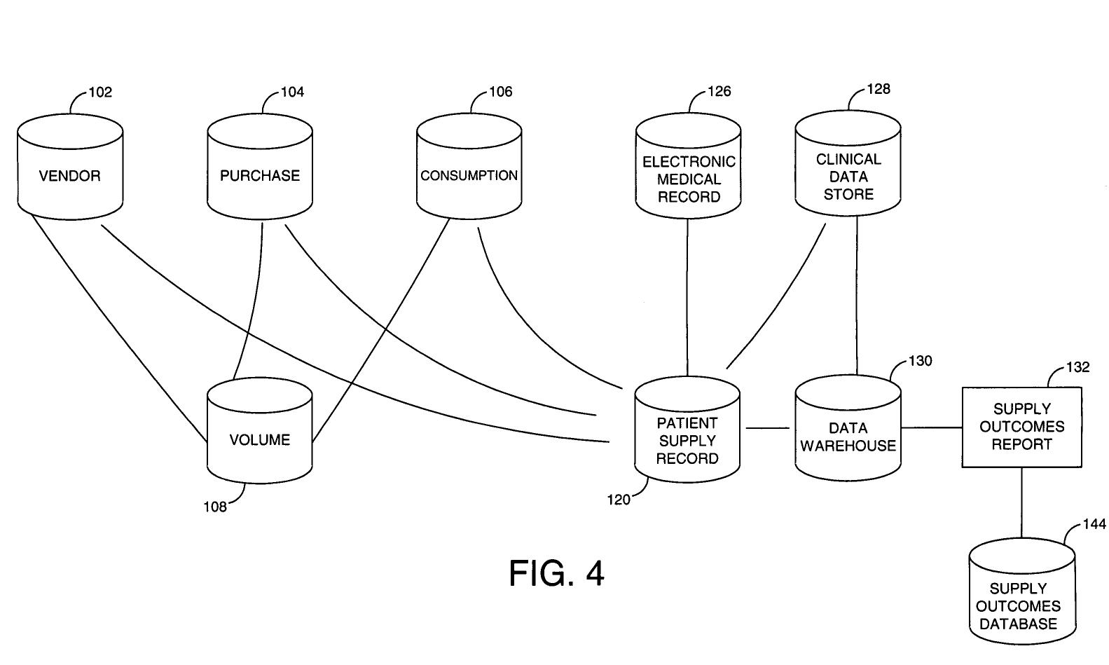

FIG. 4 illustrates an architecture in which a supply management platform including an ability to view supply outcomes may operate, according to an embodiment of the invention. As shown the information network may likewise include a vendor database 102, purchase database 104, consumption database 106 and volume database 108, among other resources and data links. More particularly, in embodiments as shown the clinical data store 128 recording patient medical information along with the patient supply record 120 and other data stores may communicate with a data warehouse 130, which may be or include, for example, a data hosting facility such as SQL databases, storage and associated query engines.

As shown in this implementation, an administrator or other user may interrogate the data warehouse 130 to extract analytics regarding patient outcomes as a function of supply selections and use, in the form of a supply outcomes report 132 or other output. That is, according to embodiments of the invention as shown, managerial or other users may generate reports against clinical outcomes data, such as recovery times, surgical infections or other complications or other measures, and the actual clinical supplies used in the care of a given patient or patients leading or contributing to those outcomes or results. A user may query those or other clinical data resources to determine, for instance, the mean survival time for patients receiving an antimicrobial-coated stent or infection rate for patients receiving an orthopedic prosthesis or a certain type or manufacture.

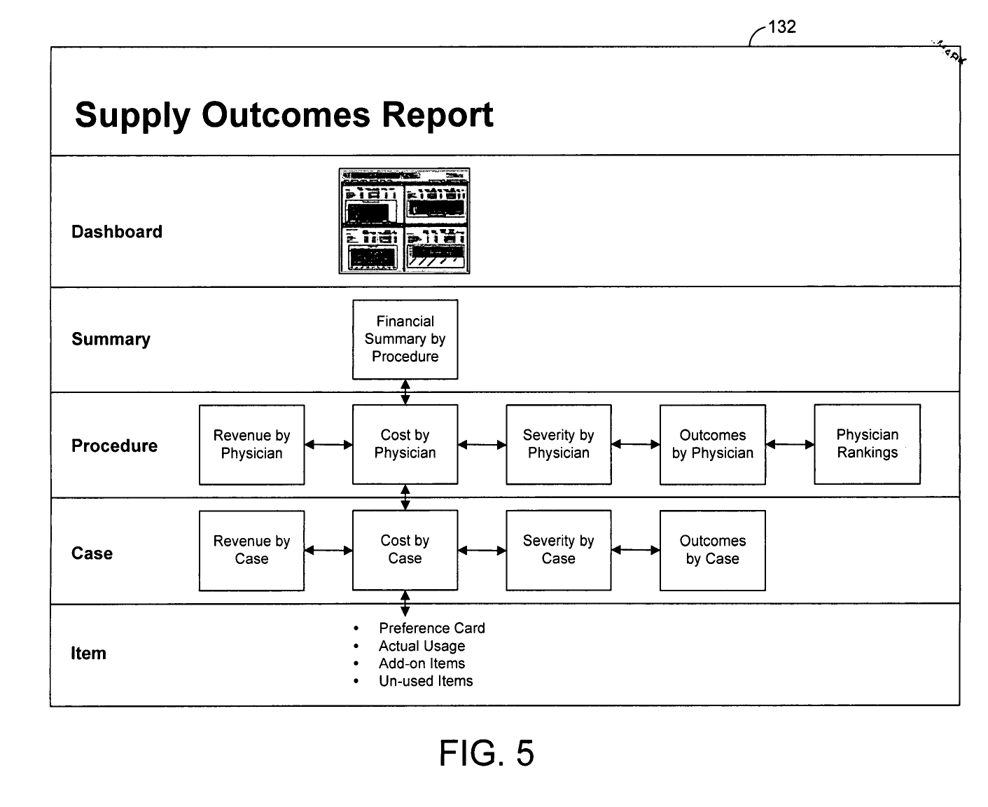

FIG. 5 shows an illustrative supply outcomes report 132, which decomposes analytics at various levels as a function of supply selections and other clinical and other variables. An administrator may for instance operate a dashboard or other graphical or other user interface to execute queries, run reports and interrogate data stores such as supply outcomes database 144, clinical data store 128, vendor database 102, purchase database 104, consumption database 106, electronic patient record 126 or other databases or other data sources. As shown the operator may drill into various levels of detail including financial summary level, procedure-level costing, outcomes, case severity, physician results and rankings and other data or output. Likewise the user may elect to examine various clinical and financial details at a case level, as well as review at the lowest illustrated level individual supply items or supply records such as preference card 114, records of actual usage, add-on or unused material. Other levels and content of the analysis presented in supply outcomes report 132 are possible.

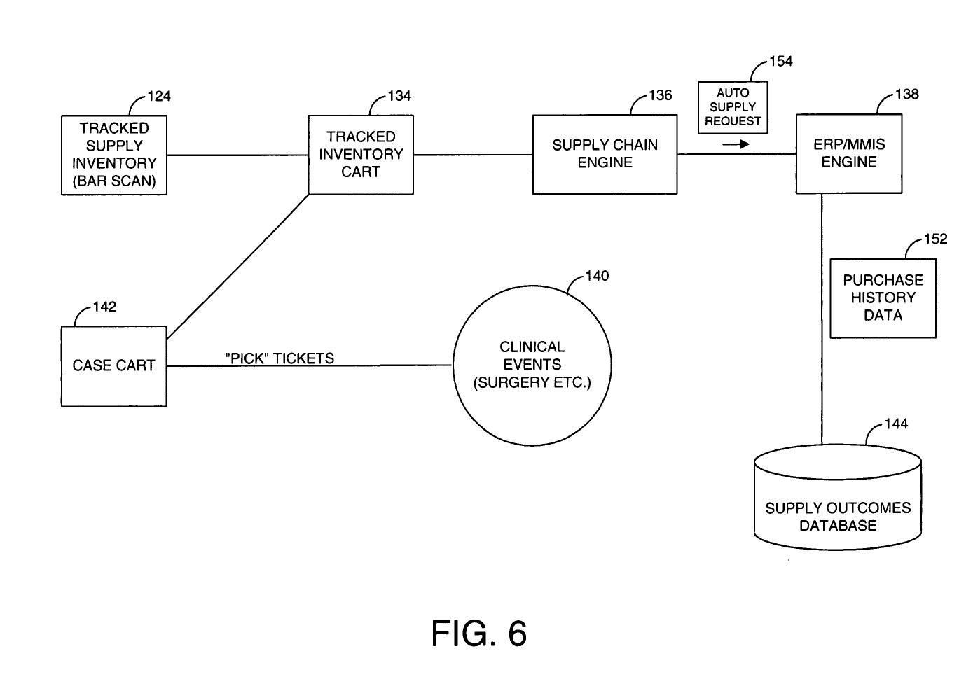

FIG. 6 illustrates an architecture in which a supply management platform may operate, according to further embodiments of the invention. In the illustrative scenario, patients may receive clinical care during a clinical event 140, such as a surgical or dental procedure, during which physicians or other care providers may issue or select pick tickets or other indicators of desired supplies and material for the clinical service they are performing. As shown, the pick ticket or other selection indicator may be conveyed to or fulfilled by clinical supplies housed for instance in a case cart 142, such as a surgical instrument tray or cart. The supplies arrayed in case cart 142 may be provided from a tracked inventory cart 134, which for instance is stocked with tracked supply inventory 124, such as supplies and material encoded via bar code scan, RFID, manual entry or other techniques. The actual consumption of physical supplies may therefore in embodiments be tracked while the clinical event 140 is carried out, in realtime or substantially realtime, or in later administrative processing. The consumption may be documented on the preference card or by other clinical documentation known to those of skill in the art.

In embodiments as shown, the tracked inventory cart 134 may likewise communicate a state of clinical supply inventory to a supply chain engine 136, for instance to report quantities, condition, freshness or other data about instruments, diagnostic equipment, medications or other disposable or non-disposable supplies to that engine. Supply chain engine 136 may be configured, for instance, with a set of rules for evaluating the condition and status of the clinically available supplies reported in that fashion. Supply chain engine 136 may, for example, be programmed to detect the quantity of a given supply reaching a certain threshold, upon which actions to resupply the clinical store may be automatically taken.

Specifically supply chain engine 136 may generate an automatic supply request 154 when low reserve quantity of a given supply or other triggering criteria are reached. The automatic supply request 154 may in embodiments be communicated to other information resources in the organization, as illustrated to an enterprise resource planning/medical management information system (ERP/MMIS) engine 138. The ERP/MMIS engine 138 may process the automatic supply request 154, for instance to communicate a supply purchase order or other procurement document, file or transmission to a vendor, manufacturer or other supplier of clinical materials. In embodiments, supply chain engine 136 may, depending on programmed rules, accumulate orders before generating automatic supply request 154 for given types or categories of supplies to satisfy in batch or aggregate fashion, for instance to derive favorable purchase price or other terms, or when the order is for non-critical or non-time sensitive material.

In embodiments as shown, the ERP/MMIS engine 138 or other information infrastructure responding to automatic supply request 154 may also communicate purchase history data 152 concerning the supply order or other action to supply outcomes database 144, so that clinical outcomes may be recorded or associated with ongoing supply procurement. Other purchase or transaction processing is possible.

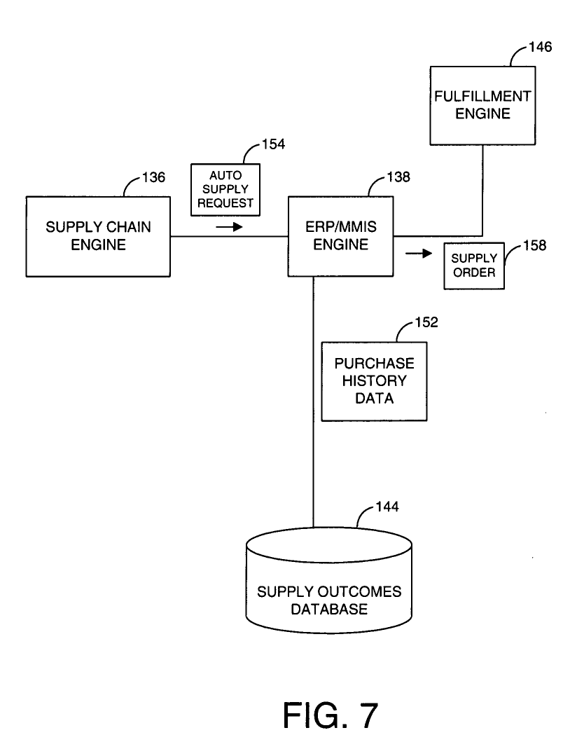

FIG. 7 illustrates an architecture in which a supply management platform may operate, according to further embodiments of the invention. In this illustrative scenario, a supply chain engine 136 operating in or in conjunction with a clinically related site may communicate an automatic supply request 154 to an internal or external ERP/MMIS engine 138, which in turn transmits a supply order 158 to a fulfillment engine 146, which in embodiments may be located at or communicate with a supply vendor, such as a manufacturer or distributor of clinical supplies.

The vendor, distributor or other entity may then in embodiments execute an automated purchase fulfillment of the supplies ordered in supply order 158, for example to direct that supplies be shipped or transported from a closest or most efficient warehouse or other supply facility to the ordering facility. Accounts receivable or other billing, tracking and other information may likewise in embodiments be automatically exchanged or reconciled using business-to-business billing or other platforms. Purchase history data 152 may likewise be returned to supply outcomes database 144 or other data stores to track cost, clinical and other variables, according to embodiments of the invention.

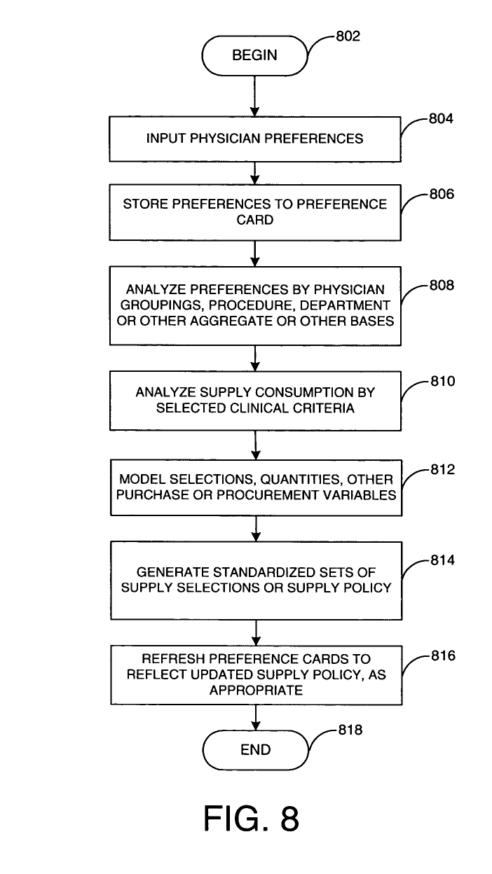

FIG. 8 illustrates a flowchart of overall supply preference and policy processing according to an embodiment of the invention. In step 802, processing may begin. In step 804, the supply preferences or selections of physicians or other care providers may be input, for example to select surgical instruments by size, grade, manufacturer or other specifications. In step 806, the preferences may be stored to a preference card 114 or other storage resource. In step 808, the supply preferences expressed by physicians or other care providers may be analyzed by practice groupings, department, procedure types, or other aggregate or other bases.

In step 810, the supply consumption of a given department or other unit of a clinically related site or sites may be analyzed by selected clinical or other criteria, for instance to determine what percentage of pediatric surgeons are using a prosthetic implant rod made by a given vendor or manufacturer, or for all hip replacement procedures which product is being selected which percentage of the time. Other criteria or reports are possible. In step 812, the actual or projected supply policy 112 or supply selections, quantities or other purchase or procurement variables may be analyzed or modeled in value analytic engine 110, for instance to determine whether standardizing on a given type of surgical stent or other supply would lead to volume pricing, delivery time or other advantages. In step 814, standardized sets of supply selections or supply policy 112 may be generated taking those analytics into account. In step 816, some or each preference card 114 for physicians and other care providers may be refreshed to reflect the updated supply policy 112. In step 818, processing may repeat, terminate or return to a prior processing point.

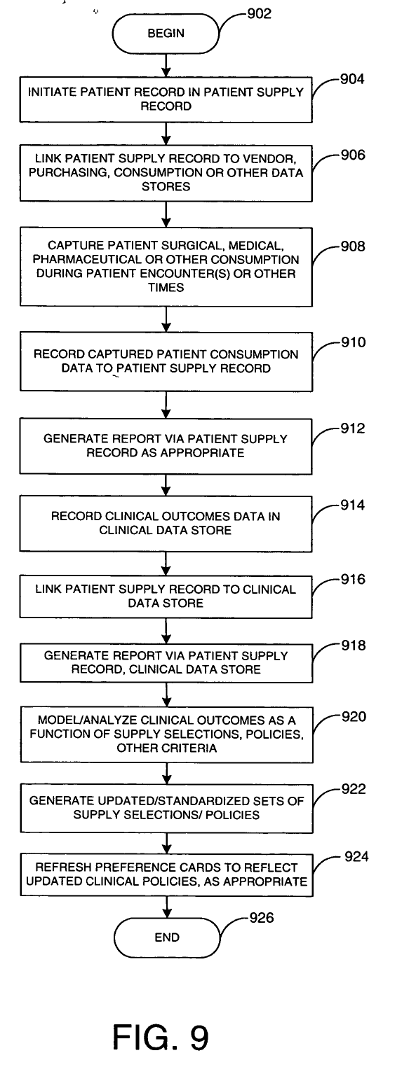

FIG. 9 illustrates overall processing of patient supply record and supply outcomes reports, according to embodiments of the invention. In step 902, processing may begin. In step 904, a patient supply record 120 may be initiated for an individual patient. In step 906, the patient supply record 120 may be linked to the vendor database 102, the purchase database 104, the consumption database 106 or other databases or stores. In step 908, the clinical supplies consumed or used by the patient during an encounter (or course or treatment) or other times may be captured, for instance by manual entry, data scan or other techniques. An encounter is defined by one or more clinical events occurring during a single visit or course of treatment. For example, an inpatient encounter may involve time in the emergency department, operating room, intensive care unit or other recovery area and patient room. During an encounter, supplies and materials such as surgical supplies and material, medical supplies such as intravenous solutions, pharmaceutical, disposable or other supplies or materiel may be used and captured and for instance temporarily stored. The clinical events (and supply consumption) may occur at any of a number of departments including but not limited to surgery, preoperative, catheter, pharmacy, radiology, laboratory and emergency departments over the period of time defining the encounter.

In step 910, the captured patient consumption data may be recorded to the patient supply record 120. In step 912, a report may be generated by queries against the patient supply record 120 as appropriate, for instance to determine how many surgical supplies a given patient has used or consumed this year. In step 914, clinical outcomes data may be generated and recorded to the clinical data store 128, for instance to memorialize the length of patient admission, vital signs at time of discharge, patient morbidity, mortality, ambulation, infectious course, prescribed medications and dose, post-operation followup, patient condition relative to initial patient condition or other clinical outcomes metrics or information.

In step 916, the patient supply record 120 may be linked to the clinical data store 128. In step 918, a report may be generated by queries against the patient supply record 120 and clinical data store 128, for instance to determine the average recovery time of patients receiving a given antibacterial stent. In step 920, clinical outcomes for one or more patients or groups of patients may be modeled and analyzed, for instance as a function of supply selections, vendors, supply policy 112, volumes, resource usage or other criteria or variables. For instance an administrator or other user may model or project the expected average duration of patient admissions if a new type of antibiotic were uniformly administered to acute pneumonia patients.

In step 922, an updated or standardized set or sets of supply selections and/or departmental or other supply policy 112 may be generated, informed by results of the clinical supply outcomes, modeling or other information. In step 924, the preference card 114 for one or more physicians or other care providers, departments or others may be updated to reflect updated supply policy 112, as appropriate. In step 926, processing may repeat, terminate or return to a prior processing point.

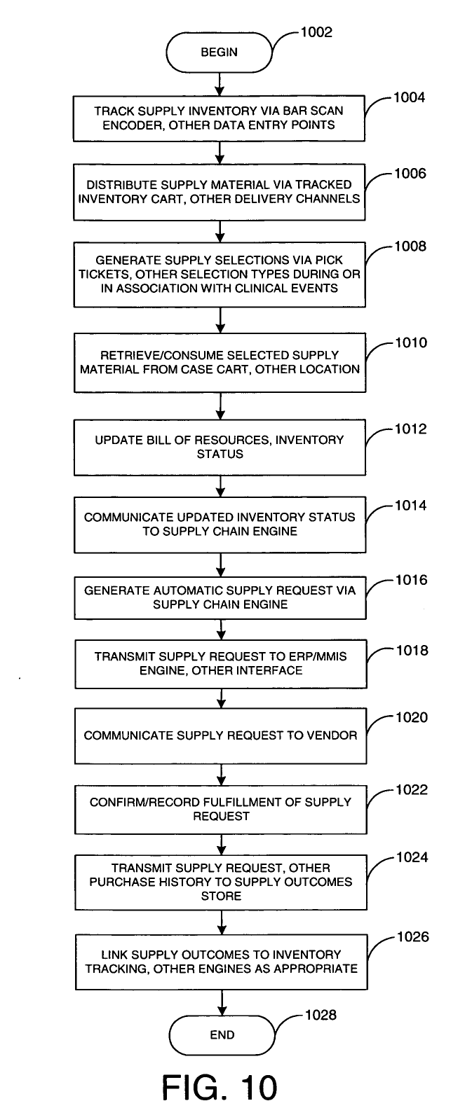

FIG. 10 illustrates overall automated supply chain ordering and supply fulfillment, according to embodiments of the invention. In step 1002, processing may begin. In step 1004, clinical supply inventory may be tracked via a bar coder scanner or other data entry points, for instance at supply closets, supply carts or other supply locations in a hospital or other clinically related site. In step 1006, supply materials such as surgical, therapeutic, pharmaceutical, disposable or other products may be distributed to or within a hospital or other clinical facility, such as via a tracked inventory card 134 or other delivery location or channel.

In step 1008, pick tickets or other selection types may be generated during or in association with clinical events, such as pick tickets generated in an operating room during a surgery. In step 1010, the selected supply or supplies may be retrieved from a case cart 142 or other location, and used or consumed in the clinical event 140 or other encounter or treatment. In step 1012, the patient supply record 112, status of supply inventory and other data may be updated to reflect the selection and use of the given supply at or during a clinical event. In step 1014, the updated inventory status may be communicated to the supply chain engine 136.

In step 1016, an automatic supply request 154 may be generated via supply chain engine 136, based for instance on the recent consumption and status of supply inventory. In step 1018, the automatic supply request 154 may be transmitted to the enterprise resource planning/medical management information systems engine 138, for instance to record order dates, amounts, vendors or other information. In step 1020, the automatic supply request 154 may be communicated to a recipient vendor, for example which request may be received on the vendor side via a fulfillment engine 146.

In step 1022, the fulfillment, shipment, delivery or other information about the physical purchase and delivery of the requested supply or supplies may be confirmed and recorded. In step 1024, the automatic supply request 154 and other purchase history data may be communicated to the supply outcomes database 144. In step 1026, the supply outcomes database may be linked to the supply chain engine 136 or other inventory tracking resources, as appropriate. In step 1028, processing may repeat, terminate or return to a prior processing point.

The foregoing description of the invention is illustrative, and modifications in configuration and implementation will occur to persons skilled in the art. For instance, while the invention has generally been described in terms of a platform in which supply preferences and policies as well as patient supply records are stored to single data stores or databases, in embodiments those and other records may be stored or hosted in multiple or distributed databases or other stores. Individual patients may in embodiments moreover have more than one associated patient supply record, within or outside a given clinically related site. Likewise, while the invention has generally been described in terms of analytics which may be generated for one clinically related site, in embodiments combined or comparative reports may be generated or combined for several related or unrelated, local or remote facilities or sites.

Further, while the invention has generally been described in terms of the capturing and processing of cost, consumption, standardization and other variables related to specifically consumable or usable supplies or equipment, in implementations the invention may also be used to capture and attribute similar or other clinical, supply, cost or other data for fixed assets or clinically related services, for instance hospital bed time, capital equipment such as diagnostic imaging machines, or other clinically related cost and supply centers.

Still further, while the invention has been described relative to a preference card or other clinical documentation to record the use of supplies during, at or after a clinical event, in an implementation of the invention the scheduling of a clinical event may be utilized to proactively affect or update inventory based on standard supplies used for the scheduled clinical events.

Other hardware, software or other resources described as singular may in embodiments be distributed, and similarly in embodiments resources described as distributed may be combined. The scope of the invention is accordingly intended to be limited only by the following claims.

1. A system for automatically generating orders for clinically related supplies, comprising:
an input interface to receive supply consumption data documented for at least one clinical event captured from at least one clinically related site; and
a supply chain engine, the supply chain engine communicating with the input interface to update supply inventory data to account for the supply consumption data and run reports against supply inventory data, and automatically generate orders for clinically related supplies based upon a state of the supply inventory data.

2. A system according to claim 1, wherein the clinically related site comprises at least one of a hospital facility, a research facility and a government facility.

3. A system according to claim 1, wherein the supply inventory data comprises at least one of clinically available quantities of surgical devices, clinically available quantities of pharmaceuticals and clinically available quantities of consumable material.

4. A system according to claim 3, wherein the supply chain engine generates a clinical supply order based upon at least one clinical quantity threshold.

5. A system according to claim 1, wherein the orders for clinically related supplies comprise purchase orders.

6. A system according to claim 1, wherein the supply consumption data comprises supply codes captured in the at least one clinically related site.

7. A system according to claim 6, wherein the supply codes comprise at least one of optically scanned bar codes, radio frequency identification codes and manually entered codes.

8. A system according to claim 1, wherein the orders for clinically related supplies are aggregated for transmission in batch mode.

9. A system according to claim 1, wherein the orders for clinically related supplies are transmitted in at least substantially realtime.

10. A system according to claim 1, wherein the orders for clinically related supplies are aggregated for a plurality of clinical departments.

11. A system according to claim 1, wherein the orders for clinically related supplies are communicated to a clinical outcomes store.

12. A system according to claim 1, wherein the orders for clinically related supplies are associated with an individual patient supply record.

13. A method for automatically generating orders for clinically related supplies, comprising:
receiving supply consumption data documented for at least one clinical event captured from at least one clinically related site;
updating supply inventory data to account for the supply consumption data;
running reports against the supply inventory data; and
automatically generating orders for clinically related supplies based upon a state of the supply inventory data.

14. A method according to claim 13, wherein the clinically related site comprises at least one of a hospital facility, a research facility and a government facility.

15. A method according to claim 13, wherein the supply inventory data comprises at least one of clinically available quantities of surgical devices, clinically available quantities of pharmaceuticals and clinically available quantities of consumable material.

16. A method according to claim 15, further comprising a step of generating a clinical supply order based upon at least one clinical quantity threshold.

17. A method according to claim 13, wherein the orders for clinically related supplies comprise purchase orders.

18. A method according to claim 13, further comprising a step of capturing supply codes captured in the at least one clinically related site.

19. A method according to claim 18, wherein the supply codes comprise at least one of optically scanned bar codes, radio frequency identification codes and manually entered codes.

20. A method according to claim 13, wherein the orders for clinically related supplies are aggregated for transmission in batch mode.

21. A method according to claim 13, wherein the orders for clinically related supplies are transmitted in at least substantially realtime.

22. A method according to claim 13, wherein the orders for clinically related supplies are aggregated for a plurality of clinical departments.

23. A method according to claim 13, further comprising a step of communicating the orders for clinically related supplies to a clinical outcomes store.

24. A method according to claim 13, further comprising a step of associating the orders for clinically related supplies with an individual patient supply record.

25. A transmissible order for clinically related supplies, the order for clinically related supplies being automatically generated according to a method of:
receiving supply consumption data documented for at least one clinical event captured from at least one clinically related site;
updating supplying inventory data to account for the supply consumption data;
running reports against the supply inventory data; and
automatically generating at least one order for clinically related supplies based upon a state of the supply inventory data.

26. An order for clinically related supplies according to claim 25, wherein the clinically related site comprises at least one of a hospital facility, a research facility and a government facility.

27. An order for clinically related supplies according to claim 25, wherein the supply inventory data comprises at least one of clinically available quantities of surgical devices, clinically available quantities of pharmaceuticals and clinically available quantities of consumable material.

28. An order for clinically related supplies according to claim 27, wherein the method further comprises a step of generating the at least one clinical supply order based upon at least one clinical quantity threshold.

29. An order for clinically related supplies according to claim 25, wherein the at least one order for clinically related supplies comprises a purchase order.

30. An order for clinically related supplies according to claim 25, wherein the method further comprises a step of capturing supply codes captured in the at least one clinically related site.

31. An order for clinically related supplies according to claim 30, wherein the supply codes comprise at least one of optically scanned bar codes, radio frequency identification codes and manually entered codes.

32. An order for clinically related supplies according to claim 25, wherein the at least one order for clinically related supplies is aggregated with other orders for transmission in batch mode.

33. An order for clinically related supplies according to claim 25, wherein the at least one order for clinically related supplies is transmitted in at least substantially realtime.

34. An order for clinically related supplies according to claim 25, wherein the at least one order for clinically related supplies is aggregated with other orders for a plurality of clinical departments.

35. An order for clinically related supplies according to claim 25, wherein the method further comprises a step of communicating the at least one order for clinically related supplies to a clinical outcomes store.

36. An order for clinically related supplies according to claim 25, wherein the method further comprises a step of associating the at least one order for clinically related supplies with an individual patient supply record.
